(19) United States
(12) Patent Application Publication
(10) Pub. No.: US 2024/0009143 A1
(43) Pub. Date: Jan. 11, 2024

(54) EXTRACTION OF SHIKONIN FROM ARNEBIA DECUMBENS ROOTS AND USE OF SHIKONIN DERIVATIVES FOR TREATING ULCERS

(71) Applicant: KUWAIT UNIVERSITY, SAFAT (KW)

(72) Inventor: LAMYA HASAN JOWHAR HAYAT, SAFAT (KW)

(21) Appl. No.: 17/859,609

(22) Filed: Jul. 7, 2022

(51) Int. Cl. A61K 31/122; B01D 11/02

(52) U.S. Cl. CPC A61K 31/122; B01D 11/0288

(57) ABSTRACT

A method of extracting shikonin derivatives from Arnebia decumbens roots can include powdering the roots of Arnebia decumbens and extracting shikonin derivatives from the powder using isooctane. The shikonin derivatives can include four compounds: deoxyshikonin (compound B); shikonin-isovalerate (compound C); acetylshikonin (compound D); and shikonin (compound E). Acetyl-shikonin (compound D) and shikonin (compound E) can be used to treat stomach ulcers and diabetic ulcers.

### BACKGROUND

### 1. Field

The disclosure of the present patent application relates to healing wounds using natural compositions and, particularly, to a method of extracting shikonin from Arnebia decumbens roots and its use in ulcer healing.

### 2. Description of the Related Art

Shikonin and its derivatives have demonstrated various therapeutic activities, with variation in effectiveness being dependent upon the plant from which shikonin is extracted. Shikonin and its derivatives extracted from Lithospermum erythrorhizon (Le) roots have been shown to be active against gram-positive bacteria such as Staphylococcus aureus, Enterococcus faecium, and Bacillus subtilis at MICs ranging from 0.30 to 6.25 mg/mL, as well as against various species of lactic acid bacteria. In contrast, they are inactive against gram-negative bacteria such as Escherichia coli, Pseudomonas aeruginosa, and Micrococcus luteus. Shikonin extracted from the roots of Arnebia decumbens exhibited great anti-bacterial activity against Escherichia coli, Pseudomonas aeruginosa and Staphylococcus aureus.

Shikonin and derivatives extracted from the roots of Alkana tinctoria demonstrated anti-oxidant properties. It is believed that the presence of the naphthoquinone moiety is essential for this activity, while the side chain possibly plays a role in the ulcer time healing. For example, the naphthoquinon (shikonin) Fraction E absorbance of UVA & UVB increased when an acetyl group was attached as Fraction D, yet the absorbance decreased when the shikonin deoxidized as Fraction B or when the isovaleryl group was incorporated as Fraction C

The anti-cancer effect of n-hexane extract from the roots of (Le) (shikonin derivatives of Le) inhibited growth of melanoma in vivo in experimentally implanted tumor in mice upon intraperitoneal injection of the extract (10 mg/kg every 3 days). The tumor inhibition ratio was determined after 21 days of treatment, resulting in reduction in tumor growth (43%) and weight (36%). Le-Shikonin induced apoptosis in B16F10 cells by activation of caspase 3.

Thus, a method for treating stomach ulcers and diabetic ulcers is desired.

### SUMMARY

A method of extracting shikonin derivatives from Arnebia decumbens roots can include powdering the roots of Arnebia decumbens and extracting shikonin derivatives from the powder using isooctane. The shikonin derivatives can include four compounds: deoxyshikonin (compound B); shikonin-isovalerate (compound C); acetylshikonin (compound D); and shikonin (compound E). Acetyl-shikonin (compound D) and shikonin (compound E) can be used to treat stomach ulcers and diabetic ulcers.

These and other features of the present subject matter will become readily apparent upon further review of the following specification.

### BRIEF DESCRIPTION OF THE DRAWINGS

FIGS. 1A-1C depict (A) the effect of 30 μg of acetylshikonin on H. pylori cells; (B) a control plate of H. pylori cells; and (C) the effect of 60 μg of acetylshikonin on H. pylori cells.

FIGS. 2A-2C depict (A) the effect of 30 μg of acetylshikonin on Candida albicans cells; (B) a control plate of Candida albicans cells; and (C) the effect of 60 μg of acetylshikonin on Candida albicans cells.

FIG. 3 depicts the effect of total extract (TE), deoxyshikonin (B), isovalerylshikonin (C), acetylshikonin (D), and shikonin (E) on the ulcers of diabetic rats. The photographs were taken to show the process of healing on days 7, 14, 21

Similar reference characters denote corresponding features consistently throughout the attached drawings.

### DETAILED DESCRIPTION OF THE PREFERRED EMBODIMENTS

A method of extracting shikonin derivatives from Arnebia decumbens (AD) roots can include powdering the roots of AD and extracting acetylshikonin derivatives from the powder using isooctane. The shikonin derivatives can include four compounds: deoxyshikonin (compound B); shikonin-isovalerate (compound C); acetylshikonin (compound D); and shikonin (compound E). A method of treating ulcers can include administering a therapeutically effective amount of a shikonin derivative to a patient in need thereof. In an embodiment, the effective shikonin derivatives include acetylshikonin (compound D) and shikonin (compound E). In an embodiment, the ulcer includes at least one of a stomach ulcer and a diabetic ulcer.

The method of extracting shikonin derivatives can include powdering the roots of the AD desert plant and extracting shikonin from the powder with isooctane to produce a total extract (TE). The TE can be fractionated by silica gel column chromatography into four compounds: (B); (C); (D); and (E). In experiment, a significant inhibitory effect of compounds (D) and (E) on Heliobacter pylori and Candida albicans was demonstrated. Further, compounds (D) and (E) caused rat diabetic ulcers to epithelialize faster and the rate of ulcer contraction to significantly increase.

A method of treating ulcers can include administering a therapeutically effective amount of a shikonin derivative to a patient in need thereof. In an embodiment, the ulcer is selected from the group consisting of stomach ulcers and diabetic ulcers. The shikonin derivative or a pharmaceutical composition including the shikonin derivative can be administered to the patient by any suitable route. The route of administration can include intranasal administration, oral administration, inhalation administration, subcutaneous administration, transdermal administration, intradermal administration, intra-arterial administration with or without occlusion, intracranial administration, intraventricular administration, intravenous administration, buccal administration, intraperitoneal administration, intraocular administration, intramuscular administration, implantation administration, topical administration, intratumor administration, and/or central venous administration. To prepare the pharmaceutical composition, the shikonin derivative or a salt thereof, as the active ingredient, is intimately admixed with a pharmaceutically acceptable carrier according to conventional pharmaceutical compounding techniques. Carriers are inert pharmaceutical excipients, including, but not limited to, binders, suspending agents, lubricants, flavorings, sweeteners, preservatives, dyes, and coatings. In preparing compositions in oral dosage form, any of the pharmaceutical carriers known in the art may be employed. For example, for liquid oral preparations, suitable carriers and additives include water, glycols, oils, alcohols, flavoring agents, preservatives, coloring agents, and the like. Further, for solid oral preparations, suitable carriers and additives include starches, sugars, diluents, granulating agents, lubricants, binders, disintegrating agents, and the like. A therapeutically effective amount of the shikonin derivatives or an amount effective to treat an ulcer, may be determined initially from the Examples described herein and adjusted for a specific desired shikonin derivative using routine methods.

The present teachings are illustrated by the following examples.

### Example 1

### Extract Preparation

Arnebia decumbens was collected from different locations in the Kuwait Desert. The red roots of the plant were shade dried, powdered in a mill, and kept in sealed containers in the shade. The powdered material was extracted with one liter of iso-octane in a continuous soxhlet apparatus. Chromatograms run on a thin layer chromatography (TLC) silica gel 60 plate using a solvent including chloroform-acetic acid-toluene [70:2:30 v/v], provided the best separation of the total extract (TE). Four different compounds having Rf=0.75, 0.63, 0.59, and 0.34 were obtained respectively.

In detail, the total extract (TE) was subjected to purification on an acid-washed silica gel column, packed in light petroleum ether and dichloromethane, to separate the compounds from each other. The activated, acid-washed silica gel [60 mesh-silica gel] was used as an adsorbent. The (TE), dissolved in a small volume of chloroform, was loaded on the column. Then, the column was immediately eluted with 2 liters of light petroleum ether. It was further eluted with petroleum ether-dichloromethane [95:5 v/v, 200 ml] and the fraction was concentrated to yield a bright red oily material. TLC gave one spot with (Rf=0.75) which was labelled as compound (B). The solvent was removed under reduced pressure on a rotary evaporator machine at 50° C. and transferred into a vial where it was dried with nitrogen gas to give crystalized fine red needles. Continuous elution of the column with light petroleum ether-dichloromethane [90:10 v/v, 200 ml] and evaporation of the solvent under reduced pressure yielded a deep red oily material. This was further purified on a series of acid-washed silica gel columns to give a pure single red spot (Rf=0.63) which is marked as compound (C). The concentrated material crystalized as dark red needles Elution of the column was continued with a mixture of light petroleum ether-dichloromethane [85:15 v/v]. A dark red fraction was obtained. After evaporation, TLC analysis showed a single red compound (Rf=0.59). Upon drying, fine red flakes were obtained, which were labelled as compound (D).

Further elution of the column by chloroform-methanol mixture [20:80 v/v] resulted in 5 fractions of equal dark red color. The pooled fractions were further purified on two newly packed acid-washed silica gel columns. Evaporation of the solvent from the pooled fractions under reduced pressure and TLC analysis showed a single reddish violet spot with an Rf=0.34. Further removal of the solvent by nitrogen gas resulted in reddish-violet flakes, which were marked as compound (E).

The four fractions were subjected to spectroscopic analysis to determine their chemical structures, including mass spectrometry data, fourier transform infrared spectroscopy (FTIR) data, and nuclear magnetic resonance spectroscopy (NMR). Nuclear magnetic resonance spectra (NMR) was recorded on AVANCE II Bruker with a working frequency of 600 MHz for protons NMR as CDCl₃ solutions. The chemical shifts of NMR spectra were recorded in ppm scale with tetramethylsilane (TMS) as an internal standard. The following symbols have been used in tabulating the data (s) singlet; (d) doublet; (t) triplet and (m) multiplet. Organic solvents were redistilled before use.

The extract provided the following fractions: deoxyshikonin (B), isovalerylshikonin (C), acetylshikonin (D), and shikonin (E). Fractions (D) and (E) had the capacity of absorbing 95-97% of UVA & UVB.

### Example 2

### Ulcer Healing

The wound healing property of the four shikonin derivatives extracted from the roots of Arnebia decumbens on diabetic rat ulcers was investigated. Specific pathogen-free male albino rats of Wistar strain (weight, 200 to 270 g; age, 6 to 8 wk) were obtained from the animal house in Kuwait University, College of Science, and kept in an approved animal care section. The rats were maintained in micro-isolation caging in a room with controlled humidity (60%) and temperature (21° C.), a 12:12-h light:dark cycle, and free access to pelleted rodent chow and filter-sterilized water. Rats were housed individually after thigh hair removal. Diabetes was induced using streptozotocin (60 mg/Kg). The urine was tested for glucose presence before causing ulcer formation on the back tail starting point of the rat.

Animals were weighed and their fasting blood glucose levels were determined before inducing diabetes.

Diabetes Mellitus (DM) was induced chemically. After a 12 hour fast, rats received a single intraperitoneal injection of streptozotocin (60 mg/kg) (Sigma) in 0.1 M sodium tri citrate buffer (pH 4.5). Control animals were injected with 0.1 M sodium tri citrate buffer. Fasting blood glucose was measured three days later to confirm the diabetic status of the animals. For blood glucose measurements, blood was drawn from the tail vein. Blood glucose measurements were repeated 7 days after the injection. The glucose levels increased drastically indicating the induction of diabetes. Rats whose fasting blood glucose levels exceeded 250 mg/dL (13.9 mmol/dL) were considered diabetic. Water intake and weight were monitored throughout the study. The rat's blood glucose concentrations were measured using one touch-Ultra Easy™, LifeScan Inc., Milpitas, CA 95035, USA. On day 7 after the injection, the back of the right thigh hair of the 24 diabetic rats was shaved with an electric shaver. Rats then were divided into 6 groups of 4 rats each and the tested compounds were topically applied to the rats. As set forth in Table 1, Group D rats were administered deoxyshikonin in petroleum jelly (200 mg was used with D1 and D2; 400 mg with group D3 and D4). Group I rats were adminstered isovalerylshikonin in petroleum jelly (200 mg was used with group I1 and I2; 400 mg with group I3 and I4). Group A rats were administered acetylshikonin in petroleum jelly (200 mg was used with Group A1 and A2); 400 mg with group S3 and S4); Group T rats were administered total extract/crude extract in petroleum jelly (200 mg was used with Group T1 and T2; 400 mg with Group T3 and T4), and all Group C rats (control) were administered petroleum jelly in order to test the dose effect.

TABLE 1

| | | | Total vol. (ml) | Total vol. (ml) | | |
|---|---|---|---|---|---|---|
| | | | needed for each | needed for each | | |
| | | Dose of STZ to | rat from an | rat from an | Blood ‘G’ | Blood ‘G’ |
| | | induce DM (mg) | assumed stock | assumed stock | level before | level after |
| | | (65 mg STZ/ | of Na+ citrate | of Na+ citrate | induction of | induction of |
| | Rat | 1000 g rat | buffer (25 mg/ | buffer (25 mg/ | DM | DM (on day |
| Compound | wt. (g) | wht.) | 1 ml) | 1 ml) | (mmol/L) | 7) (mmol/L) |
| DA1 (200 mg) | 224 | 15.0 | 0.6 | 0.2 | 8 | 20 |
| IA1 (200 mg) | 225 | 15.0 | 0.6 | 0.2 | 8.2 | 22 |
| AA1 (200 mg) | 218 | 14.0 | 0.6 | 0.2 | 7.2 | 22 |
| SA1 (200 mg) | 240 | 16.0 | 0.6 | 0.2 | 7 | 25 |
| TA1 (200 mg) | 270 | 18.0 | 0.6 | 0.3 | 7.1 | 20 |
| DA2 (200 mg) | 210 | 14.0 | 0.6 | 0.3 | 8.3 | 18 |
| IA2 (200 mg) | 231 | 15.0 | 0.6 | 0.2 | 7.4 | 23 |
| AA2 (200 mg) | 230 | 15.0 | 0.6 | 0.2 | 9.1 | 22 |
| SA2 (200 mg) | 261 | 17.0 | 0.7 | 0.3 | 7.5 | 27 |
| TA2 (200 mg) | 253 | 16.0 | 0.6 | 0.2 | 8.6 | 21 |
| DB3 (400 mg) | 242 | 16.0 | 0.6 | 0.2 | 9 | 20 |
| IB3 (400 mg) | 220 | 14.0 | 0.6 | 0.2 | 8.8 | 20 |
| AB3 (400 mg) | 224 | 15.0 | 0.6 | 0.2 | 7.7 | 22 |
| SB3 (400 mg) | 217 | 14.0 | 0.6 | 0.2 | 6.9 | 19 |
| TB3 (400 mg) | 226 | 15.0 | 0.6 | 0.2 | 7.9 | 22 |
| DB4 (400 mg) | 200 | 13.0 | 0.5 | 0.2 | 8 | 23 |
| IB4 (400 mg) | 200 | 13.0 | 0.5 | 0.2 | 8.5 | 19 |
| AB4 (400 mg) | 220 | 14.0 | 0.6 | 0.2 | 9.3 | 20 |
| SB4 (400 mg) | 216 | 14.0 | 0.6 | 0.2 | 7.2 | 24 |
| TB4 (400 mg) | 212 | 14.0 | 0.6 | 0.2 | 8.3 | 24 |
| C1 | 241 | 16.0 | 0.6 | 0.2 | 7 | 25 |
| C2 | 244 | 16.0 | 0.6 | 0.2 | 7.4 | 22 |
| C3 | 219 | 14.0 | 0.6 | 0.2 | 9 | 22 |
| C4 | 231 | 15.0 | 0.6 | 0.2 | 8.8 | 20 |
| Total | | 332 mg | 14.3 (~15 ml) | 5.1 ml (~6 ml) | | |

All surgical procedures were performed in a sanitized surgery room by using autoclave-sterilized instruments. Because the procedures were repeated in multiple rats, two sets of instruments were used. Between uses, instruments were cleaned thoroughly to remove all organic debris, disinfected with 70% isopropyl alcohol, and re-sterilized by autoclaving. The surgeon wore clean scrubs, mask, hair cap, and sterile gloves for each rat.

The rats were anesthetized by contacting with ether (Analar-grade, SIGMA-ALDRICH®) for about two minutes prior to wounding.

On day 7 after injection, the back of all rats was thoroughly rinsed with sterile saline followed by disinfection with 10% povidone-iodine solution and then by 70% isopropyl alcohol. A sterile scalpel then was used to create a wound in the lower back of each rat. The wounds were left undressed and exposed to the environment. Animals were closely observed for infection. A photograph of the wound was taken from a 3 cm height (ES65 digital camera, Samsung, Beijing, China). Then, rats were placed in individual cages. Photographs of ulcers were taken on days 7, 14, and 21 after injection.

On day 14 after injection, a scab, defined as a crust of dried blood, serum, and exudate, was noted over each wound. The ulcer was debrided by simple mechanical removal of the scab using 70% isopropyl alcohol. The entire procedure (debridement) was repeated on day 21 after injection.

Each compound was prepared in ointment form for application on the induced wounds. 200 mg and 400 mg of each compound were dissolved in 5 ml of 70% ethanol and then dispersed in 2 g of petroleum jelly (base).

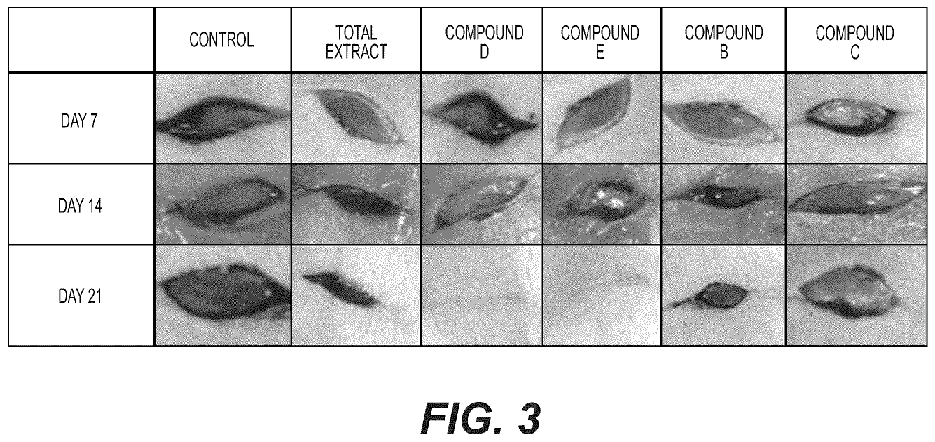

The treatment was applied topically on the wound in all cases mentioned in Table 1 on a daily basis (2 ml volume total), starting from day 14 to day 21 (7 days of induced DM). Photographs were taken on days 7, 14 and 21 of induced DM. FIG. 3 shows photographs of treated diabetic ulcers with TE and the four derivatives on days 7, 14, and 21.

### Example 3

### Anti-Bacterial

The biological activity of the 4 shikonin derivatives were tested against 2 microorganisms Heliobacter pylori (H. pylori or HP) and Candida albicans (C. albicans). Selectively, two of the 4 components (D: Acetylshikonin and E: Shikonin) had an inhibitory effect.

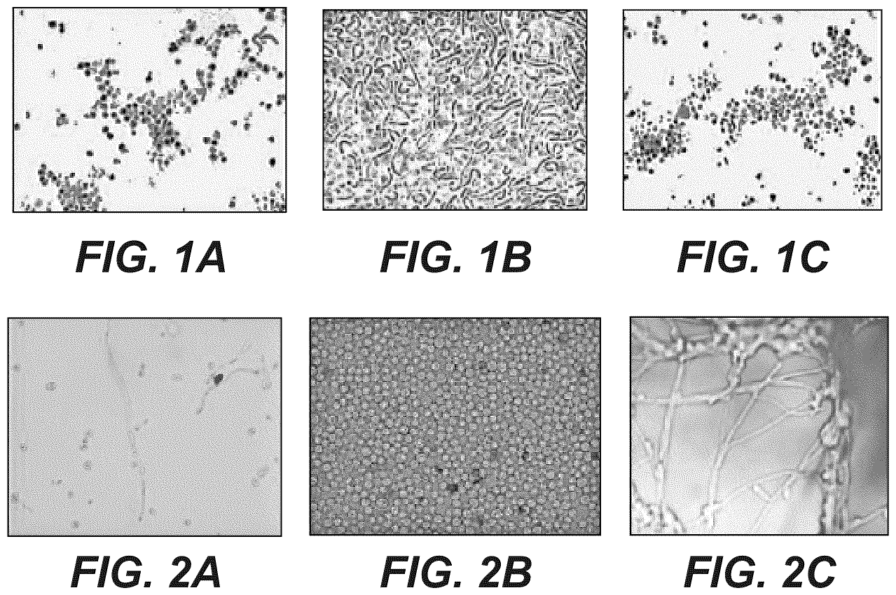

In vitro testing of acetylshikonin on H. pylori cells resulted in morphological changes to the cells (FIGS. 1A-1C). In particular, the cells changed from rod shape into a coccoidal shape. A more significant effect was observed with 60 μg than with 30 μg. In vitro testing of acetylshikonin on Candida albicans cells resulted in morphological changes to the cells (FIGS. 2A-2C). In particular, the cells changed from ovoid into a filamentous shape. A more significant effect was observed with 60 μg than with 30 μg.

Gastric ulcer caused by H. pylori is conventionally treated by a triple therapy (Amoxicillin 1-2 g. Clarithromycin 1 g, & omeprazole 80 mg/day; dose=3080 mg/day) with a reported 70% inhibition and deleterious gastric side effects. The two shikonin derivatives (compounds D and E) had an inhibition of 41.6% associated with H. pylori, with a dose of 0.06 mg/day. Hence, compounds D and E could successfully replace the triple therapy with no deleterious side effects.

It is to be understood that the method of extracting shikonin from Arnebia decumbens is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

We claim:

1. A method of obtaining shikonin derivatives, comprising:
obtaining roots of Arnebia decumbens;
preparing a powder from the roots of the Arnebia decumbens; and
extracting shikonin derivatives from the powder.

2. The method as recited in claim 1, wherein the shikonin derivatives are extracted with isooctane using a Soxhlet apparatus.

3. The method as recited in claim 2, wherein the shikonin derivatives comprise deoxyshikonin, shikonin-isovalerate, acetylshikonin, and shikonin.

4. A method of treating ulcers, comprising administering a therapeutically effective amount of the shikonin derivatives obtained according to the method of claim 1 to a patient in need thereof.

5. The method of treating ulcers according to claim 4, wherein the shikonin derivatives are selected from the group consisting of deoxyshikonin, shikonin-isovalerate, acetylshikonin, and shikonin.

6. The method of claim 4, wherein the shikonin derivatives are administered topically.

7. The method of claim 4, wherein the shikonin derivatives are administered orally.

8. The method of claim 4, wherein the ulcers are selected from the group consisting of diabetic ulcers and stomach ulcers.

9. A method of preparing shikonin derivatives, comprising:
obtaining roots of Arnebia decumbens;
preparing a powder from the roots of the Arnebia decumbens; and
extracting shikonin derivatives from the powder, the shikonin derivatives comprising deoxyshikonin, shikonin-isovalerate, acetylshikonin, and shikonin.

10. The method of claim 9, wherein the shikonin derivatives are extracted with isooctane using a Soxhlet apparatus.

11. A method of treating ulcers, comprising administering a therapeutically effective amount of the shikonin derivatives prepared according to the method of claim 8.

12. The method of claim 11, wherein the shikonin derivatives are selected from the group consisting of deoxyshikonin, shikonin-isovalerate, acetylshikonin, and shikonin.

13. The method of claim 11, wherein the shikonin derivatives are topically administered.

14. The method of claim 11, wherein the shikonin derivatives are orally administered.

15. The method of claim 11, wherein the ulcers are selected from the group consisting of diabetic ulcers and stomach ulcers.

16. A method of treating ulcers, comprising administering a therapeutically effective amount of a shikonin derivative to a patient in need thereof.

17. The method of claim 16, wherein the shikonin derivatives are selected from the group consisting of deoxyshikonin, shikonin-isovalerate, acetylshikonin, and shikonin.

18. The method of claim 16, wherein the shikonin derivatives are topically administered.

19. The method of claim 16, wherein the shikonin derivatives are orally administered.
